(19) United States
(12) Patent Application Publication
(10) Pub. No.: US 2014/0171752 A1
(43) Pub. Date: Jun. 19, 2014

(54) APPARATUS AND METHOD FOR CONTROLLING EMOTION OF DRIVER

(71) Applicant: Electronics and Telecommunications Research Institute, Daejeon (KR)

(72) Inventors: Byoung-Jun PARK, Iksan (KR); Sang-Hyeob KIM, Daejeon (KR); Eun-Hye JANG, Chungcheongnam-do (KR); Chul HUH, Daejeon (KR); Myung-Ae CHUNG, Daejeon (KR)

(73) Assignee: Electronics and Telecommunications Research Institute, Daejeon (KR)

(21) Appl. No.: 14/020,572

(22) Filed: Sep. 6, 2013

(30) Foreign Application Priority Data
Dec. 14, 2012 (KR) 10-2012-0146418

(51) Int. Cl. A61B 5/16

(52) U.S. Cl. CPC A61B 5/165
USPC 600/301

(57) ABSTRACT

An apparatus for controlling emotion of a driver includes an emotion sensor unit configured to collect a biomedical signal from the driver, and generate biomedical information data based on the collected biomedical signal, a user memory unit configured to store driver information that includes biomedical signals for respective emotional states of the driver and a plurality of correspondence contents, and deliver the driver information and the correspondence content in response to a received request, and an emotion management unit configured to determine the emotional state of the driver from the driver information received from the user memory unit and the biomedical information data received from the emotion sensor unit, request a correspondence content corresponding to the determined emotional state of the driver from the user memory unit, and provide the driver with the content received from the user memory unit.

### CROSS-REFERENCE TO RELATED APPLICATION

This application claims the benefit under 35 U.S.C. §119(a) of a Korean Patent Application No. 10-2012-0146418, filed on Dec. 14, 2012, in the Korean Intellectual Property Office, the entire disclosure of which is incorporated herein by reference for all purposes.

### BACKGROUND

1. Field

The following description relates to technology for emotion recognition, and more particularly, to an apparatus and method for analyzing and recognizing emotion of a human from a biomedical signal.

2. Description of the Related Art

The conventional vehicle related technologies have been developed into technology for running the vehicle faster and technology for reducing fuel, while the recent vehicle technology has been developed to improve the safety and convenience of a passenger. Accordingly, various technologies have been developed and applied to vehicles to ensure the safety of a passenger in an accident. The structural characteristics of a vehicle are important to the safety of vehicles, but the emotional state of a driver also has a great influence on the vehicle safety. However, the safety related technologies applied to the vehicles today do not consider the emotional state of a driver.

If the emotional state of a driver is abruptly changed without remaining in a normal state while driving, the power of attention and the judgments of the driver may be instantly lowered or lost, thereby increasing the risk of an accident. In addition, if the emotional state of the driver turns to a drowsy state, a fatal accident may be caused. As such, for the safety of a driver, the emotional state of the driver needs to be recognized, and while driving, the emotion needs to be adjusted according to the recognized emotional state of the driver. For measuring the emotion of a human, methods of analyzing a facial expression, an eye movement, an emotional voice, and a biomedical signal of a human are used.

### SUMMARY

The following description relates to an apparatus and method which are capable of recognizing and analyzing an emotional state of a driver by measuring a facial expression, a voice and a biomedical signal through a steering wheel of a vehicle, and adjusting the emotion of the driver to keep the emotional state with a normal state.

In one general aspect, an apparatus for controlling emotion of a driver includes an emotion sensor unit, a user memory unit, and an emotion management unit. The emotion sensor unit may be configured to collect a biomedical signal from the driver, and generate biomedical information data based on the collected biomedical signal. The user memory unit may be configured to store driver information that includes biomedical signals for respective emotional states of the driver and a plurality of correspondence contents, and deliver the driver information and the correspondence content in response to a received. The emotion management unit may be configured to determine the emotional state of the driver based on the driver information received from the user memory unit and the biomedical information data received from the emotion sensor unit, request a correspondence content corresponding to the determined emotional state of the driver from the user memory unit, and provide the driver with the content received from the user memory unit.

The emotion sensor unit may include an image recognition apparatus to recognize a face, a gesture and a state of a pupil of the driver, a voice recognition apparatus to recognize a voice of the driver, and a contact sensor. The contact sensor may include a skin temperature measuring device, a pulse wave measuring device, a skin conductivity measuring device, an electrocardiogram (ECG) measuring device and a photoplethysmography (PPG) measuring device. The contact sensor may be located on a steering wheel of a vehicle, and collect the biomedical signal while being in direct contact with hands of the driver.

The emotion management unit may include an emotion analysis unit configured to determine the emotional state of the driver by comparing the received biomedical information data with the received driver information, and to transmit emotional state information based on the determined emotional state of the driver; and an emotion control unit configured to search for a correspondence content among the plurality of correspondence contents to adjust the determined emotional state of the driver to a normal state, based on the received emotional state information, and to provide the found correspondence content to the driver. The emotion control unit may adjust the emotional state of the driver by at least one of methods of providing an image content through an image playback apparatus connected to a vehicle, providing a voice content through a voice playback apparatus, controlling a lighting, controlling an air conditioner, and opening/closing a window.

In another general aspect, a method of controlling emotion of a driver is achieved as follows. First, a biomedical signal may be collected from the driver. An emotional state of the driver may be determined based on the biomedical signal collected from the driver and driver information that includes biomedical signals for respective emotional states of the driver. Thereafter, the emotional state of the driver may be adjusted by searching for a correspondence content based on the determined emotional state of the driver, and providing the driver with the found correspondence content. In addition, it may be monitored whether the emotional state of the driver receiving the correspondence content is recovered to a normal state, and information about evaluating the correspondence content provided to the driver and the biomedical signal of the driver may be updated if the emotional state of the driver is recovered. Thereafter, it may be determined whether the driver is registered, by comparing the biomedical signal collected from the driver with the driver information.

Other features and aspects will be apparent from the following detailed description, the drawings, and the claims.

### BRIEF DESCRIPTION OF THE DRAWINGS

FIG. 1 is a block diagram illustrating an apparatus for controlling emotion of a driver in accordance with an example embodiment of the present disclosure.

FIG. 2 is a detailed block diagram illustrating an apparatus for controlling emotion of a driver in accordance with an example embodiment of the present disclosure.

FIG. 3 is a flowchart showing a method of controlling an emotion of a driver in accordance with an example embodiment of the present disclosure.

FIG. 4 is a diagram illustrating an example of a steering wheel applied with the apparatus for controlling the emotion of the driver in accordance with the present disclosure.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

The following description is provided to assist the reader in gaining a comprehensive understanding of the methods, apparatuses, and/or systems described herein. Accordingly, various changes, modifications, and equivalents of the methods, apparatuses, and/or systems described herein will suggest themselves to those of ordinary skill in the art. Also, descriptions of well-known functions and constructions may be omitted for increased clarity and conciseness. In addition, terms described below are terms defined in consideration of functions in the present invention and may be changed according to the intention of a user or an operator or conventional practice. Therefore, the definitions must be based on content throughout this disclosure.

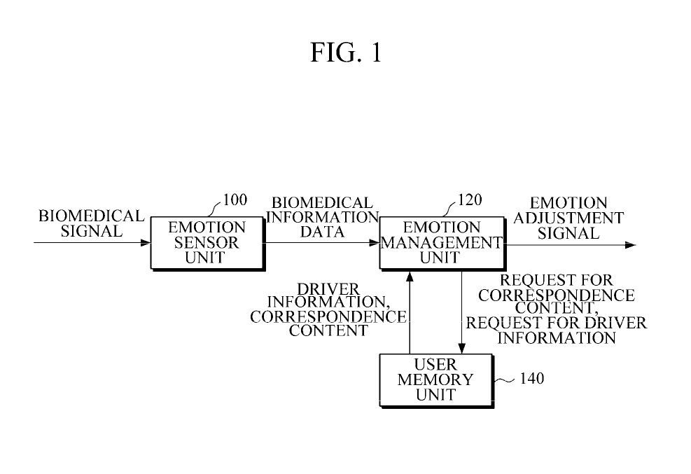

FIG. 1 is a block diagram illustrating an apparatus for controlling emotion of a driver in accordance with an example embodiment of the present disclosure.

Referring to FIG. 1, an apparatus for controlling an emotion of a driver may include an emotion sensor unit 100, an emotion management unit 120 and a user memory unit 140.

The emotion sensor unit 100 may include a voice sensor to collect a voice signal generated from the driver, an image sensor to collect an image signal, for example, a facial expression, a state of an eyeball and pupil, and a gesture, etc. of the driver, and a contact sensor to measure biomedical information of the driver. The emotion sensor unit 100 may collect biomedical signals from the driver using the above described sensors.

The biomedical signals collected from the driver may include biomedical signals collected from a face, a facial expression, a gesture, and a voice of the driver, and biomedical signals collected through direct contact with the driver. The voice sensor may collect various voice signals generated from the driver. The voice signal generated from the driver may include a voice the driver says. The image sensor may include an image pickup apparatus, photograph the driver through the image pickup apparatus, and collect the image signals including the facial expression, the state of the eyeball/pupil, or the gesture, etc. of the driver.

The contact sensor may collect the biomedical signals by being in direct contact with the body of the driver. The contact senor may be located on the surface of the steering wheel of a vehicle, and measure the biomedical signal while being in direct contact with the hands of the driver who manipulates the steering wheel. The contact sensor may include a skin temperature measuring device, a pulse wave measuring device, a skin conductivity measuring device, an electrocardiogram (ECG) measuring device, and a photoplethysmography (PPG) measuring device.

The emotion sensor unit 100 may generate biomedical information data of the driver based on the collected biomedical signals of the driver, and transmit the generated biomedical information data to the emotion management unit 120.

The emotion management unit 120 may recognize the emotional state of the driver by analyzing the biomedical information data received from the emotion sensor unit 100. The biomedical information data received from the emotion sensor unit 100 may include information about biomedical signals of the driver that include a voice, an image, a blood pressure, a heart rate, a temperature, a pulse wave, and an electrocardiogram of the driver. The emotion management unit 120 may analyze the received biomedical information data by use of various emotion recognition technologies to recognize the emotional state of the driver.

The emotion management unit 120 may receive driver information including emotion information and biomedical information of the driver from the user memory unit 140. The received emotion information and biomedical information of the driver may serve as a reference to be compared with the collected biomedical information data of the driver. The emotion management unit 120 may analyze the received biomedical information data of the driver with reference to the emotion information and the biomedical information that are stored in advance, thereby more precisely recognizing the emotional state of the driver. For example, each person may have different heart rates and blood pressures, and thus a reference of the heart rate or the blood pressure to determine whether the driver is in an excited state may be different from each driver. In this case, with reference to the emotion information and the biomedical information of each driver that are stored in advance, the emotional state of each driver is more precisely recognized.

The emotion management unit 120 may adjust the emotional state of the driver based on the recognized emotional state of the driver. If determined that the recognized emotional state of the driver has an effect on the safety of a vehicle driving, the emotion management unit 120 may search for a correspondence content that is stored in the user memory unit 140 in order to adjust the emotional state of the driver to be recovered a normal state, receive the found content, and transmit the received content to the driver. The emotional adjustment of the driver may be induced by measures of providing a content through a media apparatus, and using an air conditioner, a voice apparatus, and an image apparatus, etc. For example, if determined that the driver is in a sleeping state or enters the sleeping state, the air conditioner may be operated or a window of a vehicle may be opened so that the driver is prevented from dozing. Alternatively, an appropriate music may be provided to the driver using a media apparatus, or a direct alert may be delivered using a voice apparatus or an image apparatus.

The user memory unit 140 may store the driver information and the correspondence content, and based on a request for a correspondence content or driver information which is received from the emotion management unit 120, deliver the correspondence content and the driver information to the emotion management unit 120.

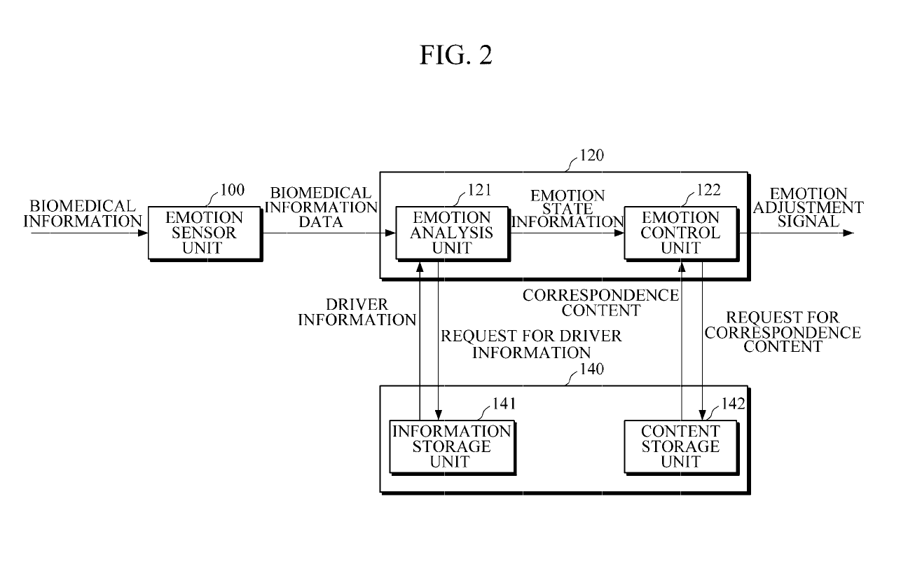

The detailed description of the emotion management unit 120 and the user memory unit 140 will be described in detail with respect to FIG. 2.

FIG. 2 is a detailed block diagram illustrating an apparatus for controlling emotion of a driver in accordance with an example embodiment of the present disclosure. Referring to FIG. 2, an emotion management unit 120 of an apparatus for controlling emotion of a driver in accordance with the present disclosure may include an emotion analysis unit 121 and an emotion control unit 122, and the user memory unit 140 may include an information storage unit 141 and a content storage unit 142.

The emotion analysis unit 121 may recognize the emotional state of the driver by analyzing the biomedical information data received from the emotion sensor unit 100. The received biomedical information data may include image information including a face, a facial expression and a gesture of the driver, voice information including a voice the driver says, and various biomedical signals measured by being in direct contact with the driver. The emotion analysis unit 121 may extract driver state information, such as a heart rate variation (HRV), a respiration rate, a pulse wave velocity (PWV), and a temperature from the biomedical information, and recognize the emotional state of the driver based on extracted various information.

The emotion analysis unit 121 may receive the driver information from the information storage unit 141. The received driver information may include biomedical information corresponding to various emotional states of the driver. The driver information may include image information including a face, a facial expression and a gesture of a driver, voice information generated from the driver, and various biomedical signals measured through contact with the driver, when the driver is in a certain emotional state. For example, biomedical information of the driver about when the driver is in an angry state, a bored state or a drowsy state may be stored in advance as the driver information.

The emotion analysis unit 121 may improve the accuracy of recognition by comparing the driver information received from the information storage unit 141 with the received biomedical information data. The biomedical signal or image/voice information representing each emotional state may be slightly different according to each person. The received driver information may be used to more precisely recognize the emotional state of the driver. For example, when a driver is in an emotion state of boredom, a face, a facial expression, a gesture and a biomedical signal of the driver are stored, and also driver state information, such as a heart rate variation (HRV), a respiration rate, pulse a wave velocity (PWV), and a temperature are stored in advance. The emotion analysis unit 121 may compare a driver information extracted the received biomedical information data with the driver information that is stored in advance, thereby more precisely recognizing the current emotional state of the driver.

The emotion analysis unit 121 may generate emotion state information including information about the emotional state of the driver, based on the received biomedical information data and the received driver information, and deliver the generated emotion state information to the emotion control unit 122.

The emotion control unit 122 may adjust the emotional state of the driver based on the emotional state information received from the emotion analysis unit 121. The emotion analysis unit 121 may recognize the current emotional state of the driver based on the received biomedical information data and the received driver information. The recognized emotional state of the driver may include various states including a normal state (i.e., a normal composure state), a bored or sleepy state, an excited state, and a distracted state. Such various emotional states of the driver may have an effect on the driving and the safety of the driver. For example, when the driver is in a bored or sleepy state, a possibility of a drowsy driving may be significantly high. During the drowsy driving, the safety of the driver is seriously threatened. Accordingly, for the safety of the driver, the emotional state of the driver may need to be controlled.

Accordingly, first, the emotion control unit 122 may determine whether the emotional state of the driver determined based on the received emotion state information has an effect on the safety of the driver. For example, if recognized that the driver is falling into a drowsy state, the emotion control unit 122 may determine that a problem for the safety of the driver has occurred. In addition, if recognized that the driver is in an excited state, the emotion control unit 122 may also determine that a problem for the safety of the driver has occurred. The reference for which the emotion control unit 122 determines whether the emotion state of the driver has an effect on the safety of the driver may include the drowsiness, excitement and distraction states that are commonly considered, and also include all the emotional states that may be recognizable depending on a driver himself or herself, or a setting during a product design process.

The emotion control unit 122, if determined that there is a problem for the safety of the driver, may determine a suitable countermeasure for adjusting the emotional state of the driver based on the received emotional state information. The method of adjusting the emotional state of the driver by the emotion control unit 122 may use all apparatuses controllable in a vehicle, such as a media apparatus, a lighting apparatus, and an air conditioning apparatus that are mounted on the inside of the vehicle. By adjusting the brightness of the lighting or operating the air conditioning apparatus, etc. of the inside of the vehicle, opening a window, or operating a media apparatus of the inside of the vehicle, a content or message may be delivered to the driver.

The emotion control unit 122, if the countermeasure for adjusting the emotional state of the driver is determined, may perform a function for controlling the emotional state of the driver. In a case in which the emotion control unit 122 determines to deliver a correspondence content or message to the driver through a media apparatus mounted on the inside of the vehicle, the emotion control unit 122 may request the correspondence content or message from the content storage unit 142. The content storage unit 142, according to the request for the correspondence content by the emotion control unit 122, may deliver the correspondence content to the emotion control unit 122.

The emotion control unit 122 receiving the correspondence content from the content storage unit 142 may provide the user with the correspondence content through the media apparatus mounted on the inside of the vehicle. The correspondence content may include a music, an image, a voice, and various signals, etc. For example, the music to which the driver frequently listens may be played through the audio apparatus mounted on the inside of the vehicle, thereby providing the driver with the music. In addition, various messages including an alert message or a message indicating the current state may be delivered to the driver through a voice playback apparatus or an image playback apparatus.

The emotion control unit 122 may adjust the emotional state of the driver through not only a provision of the corresponding content but also a use of various apparatuses in the vehicles. The emotion control unit 122 may open the window of the vehicle or operate the air conditioning apparatus in the vehicle. In addition, the lighting in the vehicle may be controlled to adjust the indoor brightness. For example, if determined that the driver is in a drowsy state, the emotion control unit 122 may open the window of the vehicle to prevent the driver from dozing. In addition, if determined that the driver is in an excited state, the lighting of the vehicle may be controlled to adjust the brightness inside the vehicle to be dark.

The emotion control unit 122 may adjust the emotional state of the driver by at least one of methods of providing the correspondence content and controlling the apparatuses in the vehicle. In addition, the emotion control unit 122 may adjust the emotional state of the driver by simultaneously using the two methods.

The information storage unit 141, if a driver information request is received from the emotion analysis unit 121, may transmit the driver information to the emotion analysis unit 121 in response to the received driver information request. The driver information may include a face, a facial expression, a gesture, and a biomedical signal for each of the various emotional states of the driver, and may further include heart rate variation (HRV), respiration, pulse wave velocity (PWV), and temperature information. When representing the same emotional state, a physical response including a face, a facial expression, a gesture, and a biomedical signal may differ from person to person. Accordingly, when the driver represents various emotional states, the face, the facial expression, the gesture and the biomedical signal, and the information with respect to the heart rate variation (HRV), respiration, pulse wave velocity (PWV), and temperature of the driver may be input and stored into the information storage unit 141 in advance. The driver information of the driver which is additionally generated may be input to the information storage unit 141. In addition, the driver may generate the driver information by measuring a signal of the driver according to the respective emotional states through the emotion sensor unit 100 of the apparatus for controlling of the emotion of the driver. In addition, the driver information may be updated by performing feedback and learning operations through repetitive measurements.

The information storage unit 141 may store driver information of not only one driver but also two or more drivers. In a case in which the driver information of the two or more drivers are stored, the driver information of each driver may be identified using an identification number that is input by the driver, or using the measured biomedical signal.

The content storage unit 142 may store various contents including a music, a voice, an image and a message. The content storage unit 142 may classify and store the respective contents according to respective drivers or according to respective emotional states. Each of the drivers may store a different content depending on his/her own tendency, hobby and habit. In addition, different contents according to various emotional states may be stored. For example, with respect to a drowsy state, a music with a fast beat may be stored, and with respect to an excited state, a music with a slow beat such as a classic music may be stored.

The content storage unit 142 may directly receive contents from the driver and store the received contents, and may download or stream contents from an external content sever using wired/wireless communication to store and provide the content.

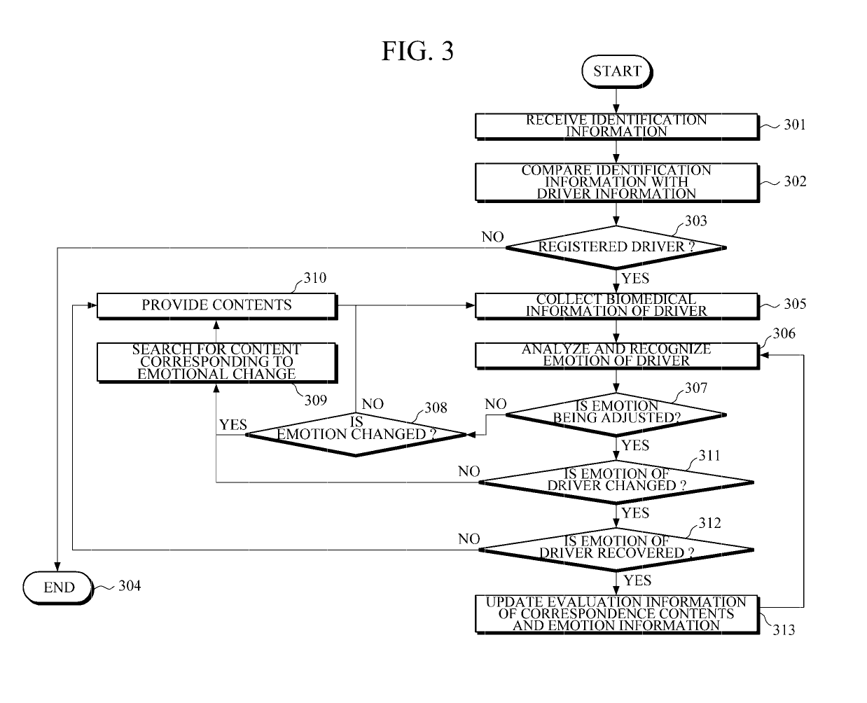

FIG. 3 is a flowchart showing a method of controlling emotion of a driver in accordance with an example embodiment of the present disclosure.

First, a method of controlling emotion of a driver in accordance with the present disclosure may receive identification information from a driver in operation 301. The identifying of the driver may be achieved commonly using two methods. In the first method, the identification information is directly received from the driver. The identification information may include an identification number or a password. In the second method, identification information may be collected by measuring biomedical signals from the driver. In the second method, when a driver sits on a driver's seat and starts driving a vehicle, the biomedical signals of the driver may be measured using various sensors included in the emotion sensor unit. In the present disclosure, biomedical information, such as the face, the gesture and the voice of the driver may be recognized using the image and the voice recognition apparatuses, or the biomedical signals of the driver may be measured using a biomedical measurement sensor included in a steering wheel when the driver grips the steering wheel.

The input identification information may be compared with registered driver information that is stored in advance in operation 302. A biomedical signal measured from the driver may be compared with the stored biomedical signal, or an input identification number may be compared with a registered identification number. Thereafter, according to the result of comparing the measured driver biomedical signal with the stored driver information, it is determined whether the corresponding driver is matched to the registered driver information in operation 303. By comparing the identification information collected from the corresponding driver with the stored driver information, it may be determined whether the corresponding driver is a registered driver or a non-registered driver. If the collected identification information is not matched to the stored driver information, the driver may be determined as a non-registered driver, and the method of controlling the emotion of the driver may be ended in operation 304.

If the measured driver biomedical signal is matched to the registered driver information as the result of comparison, biomedical information of the driver to recognize the emotional state may be collected in operation 305. The biomedical information of the driver may include signals related to the face, facial expression, state of the eyeball/pupil, gesture and voice, and also include signals generated from a skin temperature measuring device, a pulse wave measuring device, a skin conductivity measuring device, a skin conductivity measuring device, an electrocardiogram (ECG) measuring device, and a photoplethysmography (PPG) measuring device. The measuring of the biomedical signals of the driver may include a method of recognizing and measuring the driver through an image sensor and a voice sensor, and a method of measuring biomedical signals by being in direct contact with the body of the driver through two or more contact sensors included in a steering wheel.

Thereafter, the emotion of the driver may be analyzed and recognized based on the stored driver information and the measured driver biomedical information in operation 306. Even in the same emotional state, a physical response and a physical state may differ from one person to one person. Accordingly, in order to precisely recognize the emotional state of a corresponding driver from collected biomedical signals of the driver, a comparison reference and standard for the corresponding driver may be required. In the present disclosure, the measured driver biomedical information is compared with the driver information. The stored driver information may include biomedical signal information with respect to various emotional states of respective drivers. The biomedical signal or image/voice information with respect to a respective emotional state may be slightly different from person to person. The received driver information is used to more precisely recognize the emotional state of the driver. For example, when a driver is in an emotion state of boredom, the face, the facial expression, the gesture and the biomedical may be stored, and also driver state information, such as a heart rate variation (HRV), a respiration, a pulse wave velocity (PWV) and a temperature may be stored in advance. The emotion analysis unit 121 may compare the driver information extracted from the received biomedical information data with the previously stored driver information, thereby more precisely recognizing the current emotional state of the driver. The current physical state of the driver may be determined based on the measured driver biomedical information and the stored driver information, and the emotional state of the driver may be recognized through the current physical state.

Thereafter, it is determined whether the current emotional state of the driver is being adjusted in operation 307. If determined that the emotional state is not being adjusted, the emotional state of the driver may be monitored to determine whether the emotional state is changed in operation 308. In general, if the emotional state is in a normal state, it may be determined that there is no problem in the safety. However, if the emotional state may be changed from the normal state to other states, it may have a bad effect on the safety of the driver. Accordingly, the emotional state of the driver may have to be consistently monitored to find whether the emotional state of the driver is changed.

If the change in the emotional state of the driver is not detected, the biomedical signals of the driver may be continuously collected to find the emotional state (operation 305 continues).

If the change in the emotional state of the driver is detected, a content corresponding to the change of the driver emotional state may be searched in operation 309. When the change in the emotional state of the driver is detected while the emotional state is consistently monitored, a content effective to the change in the emotional state of the driver may be searched. The recognized emotional state may represent various states including a normal state, a bored or sleepy state, an excited state, and a distracted state. Such various emotional states of the driver may have an effect on a driving and a safety of the driver. For example, in a case in which the driver is in a bored or sleepy state, a possibility of a drowsy driving may be significantly high. During the drowsy driving, the safety of the driver may be seriously threatened. Accordingly, a content corresponding to the driver emotional change may be provided to control the emotion of the driver.

The correspondence content to control the emotion of the driver may include contents, such as a music, an image, a voice signal and a message, that may be provided through a media apparatus in the vehicle, and the controlling of the emotion of the driver may be achieved by using a controllable apparatus in the vehicle, for example, a lighting, a window, and an air conditioning apparatus. A suitable content corresponding to the change in the current emotional state of the driver may be searched among various contents. For example, if the driver is in a drowsy state or a bored state, a music with a fast beat or his/her favorite music may be selected, and the window may be selected to be open.

The media apparatus, the window, the lighting, and various apparatuses in the vehicle may be controlled based on the found content to adjust the emotion, to provide the driver with the found content in operation 310. In a case in which the found content is the music or the voice signal, the music may be played or the voice signal, such as an alert signal, may be played using an audio apparatus in the vehicle to provide the driver with the content. If the found content is an image, the image may be provided to the driver through an image playback apparatus in the vehicle. In addition, the brightness inside the vehicle may be adjusted by controlling the lighting in the vehicle, or opening the window of the vehicle.

If determined in operation 307 that the emotional state of the driver is being adjusted, it is determined whether the emotional state of the driver is being changed in operation 311. If the emotional state of the driver is being adjusted, it may need to be checked whether the emotional state of the driver is being changed by the provided contents. If the emotional state adjustment is attempted but the emotional state of the driver is not changed, a content corresponding to the change in the emotional state of the driver may be searched again in operation 309. In addition, the corresponding content may be evaluated depending on whether the emotional state of the driver is changed by the provided content. For example, if the emotional state of the driver is not changed, the corresponding content may be evaluated to be inappropriate for the change in the current emotional state of the driver. Such an evaluation of the content may serve as a reference when a content is searched to change the emotional state of the driver.

If the emotional state of the driver is being changed, it may be determined whether the emotional state of the driver is in a normal state in operation 312. If the emotional state of the driver is being changed, it may need to be checked whether the emotional state of the driver is recovered to the normal state, by consistently monitoring the change in the emotional state of the driver. If the emotional state of the driver is not recovered to a normal state, the content may be continuously provided in operation 310.

If the emotion of the driver turns to a normal state, the emotion information including the biomedical information of the driver and the evaluation information of the provided content are updated in operation 313. If verified that the emotion of the driver is recovered to the normal state, the measured driver biomedical signal, the recognized emotional state, and the evaluation information of the content may be updated and stored. The updated information may be used as a reference when the emotional state of the driver may be adjusted after this, and as such, the emotional state of the driver may be more efficiently adjusted.

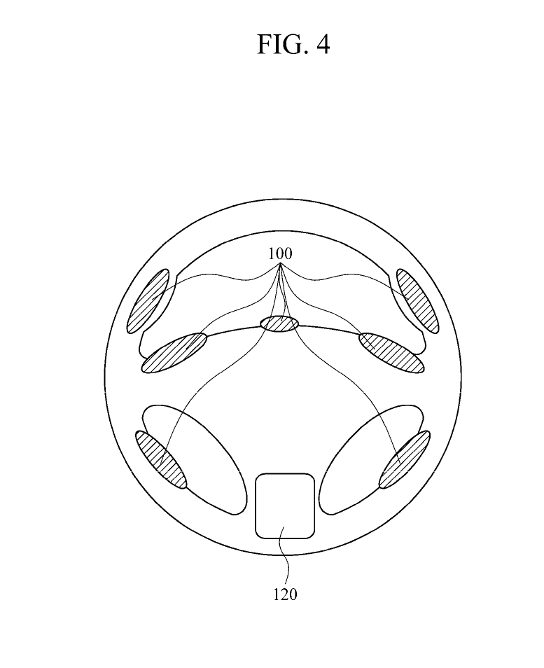

FIG. 4 is a block diagram illustrating an example of a steering wheel applied with an apparatus for controlling an emotion of a driver in accordance with the present disclosure.

Referring to FIG. 4, the apparatus for controlling the emotion of the driver in accordance with the present disclosure may be applied to a steering wheel of a vehicle. When a driver grips the steering wheel of the vehicle to drive the vehicle, the emotion sensor unit 100 located on the steering wheel may be in contact with the hands of the driver. The emotion sensor unit 100 may measure a biomedical signal of the driver through the hands of the driver being in contact with the emotion sensor unit 100. The biomedical signal of the driver collected through the emotion sensor unit 100 located on the steering wheel may be converted to biomedical information data of the driver in the emotion sensor unit 100, and the driver biomedical information may be transmitted to the emotion management unit 120. The emotion management unit 120 may recognize the emotional state of the driver based on the biomedical information data, and adjust the emotional state of the driver. In FIG. 4, the emotion sensor unit 100 may be illustrated as only having a contact sensor located on the steering wheel to collect information by being in contact with the hands of the driver, but the present disclosure may not be limited thereto. The emotion sensor unit 100 may have an apparatus for collecting a speech or image signal to be located on the steering wheel.

According to the apparatus and method for controlling the emotion of the driver of the present disclosure, the change in the emotional state of the driver occurring while driving may be recognized to detect the emotional state, such as a stressful state, an excited state or a bored state, etc. such that the emotional state of the driver is adjusted, thereby preventing from a driving mistake of the driver that may be caused in the emotional state, such as the stressful state, the excited state or the bored state, etc. and leading to a safe driving.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An apparatus for controlling emotion of a driver, the apparatus comprising:
an emotion sensor unit configured to collect a biomedical signal from the driver, and generate biomedical information data based on the collected biomedical signal;
a user memory unit configured to store driver information that includes biomedical signals for respective emotional states of the driver and a plurality of correspondence contents, and deliver the driver information and the correspondence content in response to a received request; and
an emotion management unit configured to determine the emotional state of the driver based on the driver information received from the user memory unit and the biomedical information data received from the emotion sensor unit, request a correspondence content corresponding to the determined emotional state of the driver from the user memory unit, and provide the driver with the content received from the user memory unit.

2. The apparatus of claim 1, wherein the emotion sensor unit comprises an image recognition apparatus to recognize a face, a gesture and a state of a pupil of the driver, a voice recognition apparatus to recognize a voice of the driver, and a contact sensor.

3. The apparatus of claim 2, wherein the contact sensor comprises at least one of a skin temperature measuring device, a pulse wave measuring device, a skin conductivity measuring device, an electrocardiogram (ECG) measuring device and a photoplethysmography (PPG) measuring device.

4. The apparatus of claim 2, wherein the contact sensor is located on a steering wheel of a vehicle, and collects the biomedical signal while being in direct contact with hands of the driver.

5. The apparatus of claim 1, wherein the emotion management unit comprises:
an emotion analysis unit configured to determine the emotional state of the driver by comparing the received biomedical information data with the received driver information, and to transmit emotional state information based on the determined emotional state of the driver; and
an emotion control unit configured to search for a correspondence content among the plurality of correspondence contents to adjust the determined emotional state of the driver to a normal state, based on the received emotional state information, and to provide the found correspondence content to the driver.

6. The apparatus of claim 5, wherein the emotion control unit provides the driver with the correspondence content by at least one of methods of providing an image content through an image playback apparatus connected to a vehicle, providing a voice content through a voice playback apparatus, controlling a lighting, controlling an air conditioner, and opening/closing a window.

7. A method of controlling emotion of a driver, the method comprising:
collecting a biomedical signal from the driver;
determining an emotional state of the driver based on the biomedical signal collected from the driver and driver information that includes biomedical signals for respective emotional states of the driver; and
adjusting the emotional state of the driver by searching for a correspondence content based on the determined emotional state of the driver, and providing the driver with the found correspondence content.

8. The method of claim 7, further comprising:
determining whether the driver is registered, by comparing the biomedical signal collected from the driver with the driver information.

9. The method of claim 7, further comprising:
monitoring whether the emotional state of the driver receiving the correspondence content is recovered to a normal state; and
updating information about evaluating the correspondence content provided to the driver and the biomedical signal of the driver if the emotional state of the driver is recovered.

10. The method of claim 7, wherein the biomedical signal collected from the driver comprises at least one of signals collected from a face, a gesture and a state of a pupil of the driver, and a voice sensor and a contact sensor.

11. The method of claim 10, wherein the contact sensor includes at least one of a skin temperature measuring device, a pulse wave measuring device, a skin conductivity measuring device, an electrocardiogram (ECG) measuring device and a photoplethysmography (PPG) measuring device.
